(19) United States
(12) Patent Application Publication
(10) Pub. No.: US 2012/0164729 A1
(43) Pub. Date: Jun. 28, 2012

(54) COMPOSITION FOR CULTURING PLURIPOTENT STEM CELLS AND USE THEREOF

(75) Inventor: MINORU TOMIZAWA, Yotsukaido-shi (JP)

(73) Assignee: NATIONAL UNIVERSITY CORPORATION CHIBA UNIVERSITY, Chiba-shi (JP)

(21) Appl. No.: 13/331,311

(22) Filed: Dec. 20, 2011

(30) Foreign Application Priority Data
Dec. 24, 2010 (JP) 2010-288509
Dec. 16, 2011 (JP) 2011-276111

(51) Int. Cl. C12N 5/02; C07K 14/475

(52) U.S. Cl. 435/366; 530/399; 435/325

(57) ABSTRACT

A means for feeder-free culture which can sufficiently maintain the undifferentiation potency of pluripotent stem cells such as iPS cells even without using heterologously derived cells or proteins, is provided.

Disclosed are a composition for pluripotent stem cell culture containing activin (preferably, activin A); a medium for pluripotent stem cell culture containing the composition; a method for culturing pluripotent stem cells for proliferating or establishing pluripotent stem cells such as iPS cells (preferably, mammalian iPS cells, and particularly preferably, human iPS cells) while maintaining an undifferentiated condition of the pluripotent stem cells, the method including performing the culture in the presence of activin A; and a method for preparing a pluripotent stem cell clonal population in an undifferentiated condition, the method including culturing undifferentiated pluripotent stem cells such as iPS cells (preferably, mammalian iPS cells, and particularly preferably, human iPS cells) in the presence of activin.

### BACKGROUND OF THE INVENTION

1. Field of the Invention

The present invention relates to a composition for pluripotent stem cell culture and use thereof, and more particularly, to a composition which enables pluripotent stem cell to be cultured in a medium free of supporting cells or serum, a medium containing this composition, and use of these.

2. Description of the Related Art

Pluripotent stem cells are stem cells capable of self-replication that have an ability to differentiate at least into one type each of differentiated cells that belong to ectoderm, mesoderm, and endoderm, and examples of the cells include an induced pluripotent stem cell (iPS cell), an embryonic stem cell (ES cell), an embryonic germ cell (EG cell), an embryonal carcinoma cell (EC cell), a multipotent adult progenitor cell (MAP cell), and an adult pluripotent stem cell (APS cell).

Among these, ES cells are a stem cell line produced from an embryo in the early stage of the development of an animal. Since the ES cell can be proliferated over a long time while maintaining their pluripotency by which the cell can differentiate into all kinds of tissues in vitro, application of the ES cell in the regenerative medicine is expected. However, transplantation of ES cells is accompanied by a problem of rejection, as in the case of organ transplantation. Furthermore, since the establishment of ES cells requires early embryos in the stage up to fertilized eggs or blastocysts, there is an ethical issue with the loss of life.

On the other hand, iPS cells are cells having pluripotency, which are obtained by initiating somatic cells, and the iPS cells are highly expected as ideal pluripotent cells that are free of rejection or ethical problem.

In order to culture these iPS cells in a condition that the cells retain the ability of undifferentiation maintenance, it is necessary to culture the iPS cells on a layer of supporting cells called feeder cells. As the feeder cells for culturing pluripotent stem cells such as iPS cells, use is made of, for example, fresh mouse fetal fibroblasts with their proliferative capacity having been restricted by a mitomycin treatment, and the like. However, the preparation of such feeder cells is accompanied by a problem that since the ability of undifferentiation maintenance varies with the batch, reproducibility is poor. Also, in the case where desired cells are produced from pluripotent stem cells and used in transplantation therapy, when mouse-derived fetal fibroblasts are used, there is a possibility that heterologously derived proteins may be present in mixture. Thus, there is a problem that clinical application thereof is not permissible.

In an attempt to promote a solution to the problems as described above, there have been technologies for culturing pluripotent stem cells without using feeder cells. For example, as a feeder-free medium, a feeder-free medium for primate ES cells (ReproFF) is commercially available from ReproCELL, Inc., and similarly, a serum-free medium for human ES cell maintenance (mTeSR (registered trademark) 1) is commercially available from STEMCELL Technologies, Inc.

Meanwhile, activin is a factor discovered in the follicular fluid during the process of purifying inhibin, as a protein which promotes the secretion of follicle-stimulating hormone (FSH) from the pituitary gland of a mammal. Further, activin belongs to the transforming growth factor (TGF) -β superfamily, and has a structure in which inhibin β chains (molecular weight 14,000 each) are dimerized through a disulfide bond. There are primarily three kinds of activin, and they respectively have structures of activin A (β_{A}β_{A} chains), activin B (β_{B}β_{B} chains), and activin AB (β_{A}β_{B} chains). It has been reported that among these, activin A differentiates undifferentiated embryonic cells into mesoderm or endoderm (see Smith J C, Price B M, Van Nimmen K and Huylebroeck D: Identification of a potent Xenopus mesoderm-inducing factor as a homologue of activin A. Nature, 345: 729-731, 1990; and Thomsen G, Woolf T, Whitman M, et al.: Activins are expressed early in Xenopus embryogenesis and can induce axial mesoderm and anteriorstructures. Cell, 63: 485-493, 1990). In fact, activin A is used also for human ES cells in the induction of differentiation into pancreatic β cells via endoderm (see D'Amour K A, Agulnick A D, Eliazer S, Kelly O G, Kroon E and Baetge E E: Efficient differentiation of human embryonic stem cells to definitive endoderm. Nat Biotechnol., 23: 1534-1541, 2005). On the other hand, it has also been reported that activin A maintains the undifferentiation potency of human ES cells (see Xiao L, Yuan X and Sharkis S J: Activin A maintains self-renewal and regulates fibroblast growth factor, Wnt, and bone morphogenic protein pathways in human embryonic stem cells. Stem Cells, 24: 1476-1486, 2006). At the present time, no conclusion has been drawn on whether activin A differentiates human ES cells into endoderm, or, conversely, acts in the direction of maintaining the undifferentiation potency. Furthermore, there is no report on the role of activin A in human iPS cells.

As described above, in the current situation, several media for culturing iPS cells without using feeder cells are commercially available. However, when pluripotent stem cells such as iPS cells are cultured using mTeSR (registered trademark) 1, differentiated cell groups occasionally appear, posing a problem that this medium is insufficient for maintenance of the undifferentiation potency of pluripotent stem cells. Furthermore, when pluripotent stem cells are similarly cultured using ReproFF, the cultured cells differ, in terms of the cell morphology or the like, from cells cultured using mouse-derived fetal fibroblasts as feeder cells. Thus, it has not been sufficiently verified whether ReproFF is actually capable of maintaining the undifferentiation potency. As such, in the current situation, the undifferentiation potency cannot be sufficiently maintained when pluripotent stem cells such as iPS cells are cultured using those media provided by the conventional technologies.

### SUMMARY OF THE INVENTION

Thus, it is an object of the present invention to provide a means for feeder-free culture which can sufficiently maintain the undifferentiation potency of pluripotent stem cells such as iPS cells, even without using heterologously derived cells or proteins.

The inventors of the present invention attempted, during the process of conducting studies, to form embryoid bodies (EB) from iPS cells by a hanging drop culture method in a medium containing activin A, and to differentiate the embryoid bodies into stem cells via endoderm. However, surprisingly, the inventors found that the embryoid bodies do not differentiate into endoderm, but maintain the condition of iPS cells, which retain the undifferentiation potency, even after being cultured for a long time period. That is, although the factor that maintains the undifferentiation potency of human iPS cells has been unknown heretofore, it is now made clear by the inventors of the present invention that activin A is a principal factor that maintains human iPS cells in an undifferentiated condition. Thus, the inventors of the present invention eventually completed the present invention based on this finding.

Specifically, according to an aspect of the present invention, there is provided a composition for pluripotent stem cell culture, containing activin (preferably, activin A). This composition may be a medium supplement. Furthermore, this composition can be used to proliferate pluripotent stem cells while maintaining the undifferentiated condition of pluripotent stem cells such as iPS cells (preferably, mammalian iPS cells, and most preferably, human iPS cells).

According to another aspect of the present invention, there is provided a medium for pluripotent stem cell culture containing the composition described above. It is preferable that this medium contains activin at a concentration of 3 to 30 ng/mL. Furthermore, it is preferable that this medium is free of supporting cells and/or serum, and more preferably, the medium is free of supporting cells and serum. In addition, this medium may be a cell culture minimum medium.

According to still another aspect of the present invention, there is provided a method for culturing pluripotent stem cells for proliferating or establishing pluripotent stem cells while maintaining an undifferentiated condition of pluripotent stem cells such as iPS cells (preferably, mammalian iPS cells, and particularly preferably, human iPS cells), the method including performing the culture in the presence of activin. This culturing method includes, for example, performing the culture in the medium described above.

According to still another aspect of the present invention, there is provided a method for preparing a clonal population of pluripotent stem cells in an undifferentiated condition, the method including culturing undifferentiated pluripotent stem cells such as iPS cells (preferably, mammalian iPS cells, and most preferably, human iPS cells) in the presence of activin. Similarly, there is also provided a method for preparing a clonal population of pluripotent stem cells in an undifferentiated condition, the method including isolating undifferentiated pluripotent stem cells from a living body, and culturing the undifferentiated pluripotent stem cells in the presence of activin. These preparation methods include, for example, performing the culture in the medium described above. Furthermore, these preparation methods may also include, for example, culturing a single pluripotent stem cell and thereby obtaining a clonal population thereof. Moreover, these preparation methods may include culturing pluripotent stem cells that are in lower density seeding conditions than those inducing undifferentiated proliferation of the pluripotent stem cells by the interaction between neighboring pluripotent stem cells, in the above-described medium free of supporting cells and/or serum (preferably, supporting cells and serum), and thereby obtaining a clonal population thereof.

According to still another aspect of the present invention, there is provided a use of activin (preferably, activin A) (or a composition containing activin) for culturing pluripotent stem cells while maintaining an undifferentiated condition of the cells, and proliferating or establishing the pluripotent stem cells.

According to the present invention, a means for feeder-free culture, which can sufficiently maintain the undifferentiation potency of pluripotent stem cells such as iPS cells, even without using heterologously derived cells or proteins, can be provided. More specifically, because there is no need to handle mouse-derived fetal fibroblasts as is the case in the related art, the culture of human iPS cells is made convenient and easy. The present invention is expected to be applied to the development of technologies for human iPS cell culture in the future.

### BRIEF DESCRIPTION OF THE DRAWINGS

FIG. 1 is a set of photographs illustrating the results obtained in the Example by microscopic observation of embryoid bodies formed by a hanging drop culture method from 201B7 cells cultured in the presence of activin A for the purpose of investigating the effect of the addition of activin A on the differentiation of iPS cells;

FIG. 2 is a graph illustrating the results obtained by evaluating, using an MTS assay, the proliferative capacity of 201B7 cells cultured in the presence of activin A at different concentrations;

FIG. 3 is a set of photographs illustrating the results obtained in the Example by confirming, with alkaline phosphatase (ALP) staining, that 201B7 cells cultured in the presence of activin A maintain the undifferentiation potency;

FIG. 4 is a graph illustrating the results obtained in the Example on the changes in the passage number as a result of the addition of various agents when subculture was performed by adding various agents to iPSm(−);

FIG. 5 is a set of photographs illustrating the observation images obtained in the Example by observing cells after performing subculture in the presence of various agents added to iPSm(−), with a phase contrast microscope (at the 12th passage) (left side of FIG. 5), and the observation images obtained by observing the relevant cells after treating the cells by alkaline phosphatase (ALP) staining (right side of FIG. 5). In FIG. 5, reference symbol “A” indicates activin A alone; “AC” indicates a combination of activin A+CHIR99021; and “ACL” indicates a combination of activin A+CHIR99021+LIF; and

FIG. 6 is a set of photographs illustrating the observation images obtained in the Example by observing cells obtained after performing subculture in the presence of various agents added to iPSm(−), after immunostaining the cells using several antibodies. In FIG. 5, reference symbol “A” indicates activin A alone; “AC” indicates a combination of activin A+CHIR99021; and “ACL” indicates a combination of activin A+CHIR99021+LIF.

### DETAILED DESCRIPTION OF THE PREFERRED EMBODIMENTS

The term “pluripotent” as used herein means an ability of cells to differentiate into any differentiated cells that belong to ectoderm, mesoderm, or endoderm, and an ability of cells to differentiate at least into one type each of differentiated cells that belong to ectoderm, mesoderm, and endoderm. The differentiation capacity into germ cells is also included in this concept.

“Pluripotent stem cells” mean stem cells capable of self-replication, which have an ability to differentiate into any differentiated cells that belong to ectoderm, mesoderm, or endoderm, or an ability to differentiate at least into one type each of differentiated cells that belong to ectoderm, mesoderm, and endoderm (multipotency), and specific examples include an iPS cell, an ES cell, an EG cell, an EC cell, a MAP cell, and an APS cell. “Induced pluripotent stem cells (iPS cells)”, a representative example of the pluripotent stem cells, have multipotency so that when injected into other blastocysts, the induced pluripotent stem cells can differentiate into various cells including germ cells.

“Supporting cells”, also called feeder cells, are cells which cannot proliferate per se but have metabolic activity, so that the supporting cells produce various metabolic substances and thereby help proliferation of other cells that are planted thereon. For example, in the case of iPS cells, fetal primary culture fibroblasts or STO cells, whose proliferation has been stopped by inactivating the cells, can be used as the supporting cells.

“Supporting cell-independent pluripotent stem cells” mean pluripotent stem cells capable of proliferating in the presence of serum under culture conditions that do not include supporting cells. It is essentially difficult to proliferate pluripotent stem cells by culturing while maintaining the undifferentiation potency under such culture conditions, however, by continuing successive subculture, supporting cell-independency is acquired.

The composition for pluripotent stem cell culture provided according to an aspect of the present invention is characterized by containing activin. Activin, also referred to as a follicle stimulating hormone secretion promoting protein, is a peptidic hormone having a molecular weight of about 27,000. There are three kinds of activins such as activin A, which is a homodimer of the β chain of inhibin A (β_{A}β_{A}), activin B, which is a homodimer of the β chain of inhibin B (β_{B}β_{B}), and activin AB, which is a heterodimer of the β chain of inhibin A and the β chain of inhibin B (β_{A}β_{B}). In the various monomers of activin, nine cysteine residues are very well preserved, and activin belongs to the TGF-β superfamily. The amino acid sequences of activins in vertebrates are very highly homologous, for example, between African clawed frog (Xenopus laevis) and human being, the homology of activin A is 87%, while the homology of activin B is 95%. A large number of functions of activin in living organisms are known, however, nothing is known about the function of enabling the maintenance of the undifferentiation potency of pluripotent stem cells under feeder-free culture conditions.

The amino acid sequences of activins are known, and for example, with regard to human-derived activin, reference can be made to the databases of various public institutions based on the following ID number: NM_{—}002192 (NCBI) Homo Sapiens inhibin, beta A (INHBA), mRNA.

Furthermore, the following proteins can also be similarly used as activin in the present invention:

(a) a protein having an amino acid sequence resulting from deletion, substitution, insertion, or addition of one or several amino acids of the amino acid sequence of natural activin, and having the biological activity of activin; and

(b) a protein having an amino acid sequence having at least 90% homology, preferably at least 93% homology, more preferably at least 95% homology, and even more preferably at least 99% homology, to the amino acid sequence of natural activin, and having the biological activity of activin.

These activins can be produced by any genetic engineering technique known to those having ordinary skill in the art.

The term “homology” (or “identity”) means the quantity (number) of those corresponding amino acid residues between the amino acid sequences of two polypeptide chains, which can be judged to be identical in the mutual compatibility relation between the respective groups of amino acid residues constituting the chains, and thus means the degree of sequence correlation between two polypeptide sequences or two polynucleotide sequences. This homology can be easily calculated. A number of methods for determining the homology between two polynucleotide sequences or polypeptide sequences are known, and the term “homology” is well known to those having ordinary skill in the art (for example, Lesk, A. M. (Ed.), Computational Molecular Biology, Oxford University Press, New York, (1988); Smith, D. W. (Ed.), Biocomputing: Informatics and Genome Projects, Academic Press, New York, (1993); Grifin, A. M. & Grifin, H. G. (Ed.), Computer Analysis of Sequence Data: Part I, Human Press, New Jersey, (1994); von Heinje, G., Sequence Analysis in Molecular Biology, Academic Press, New York, (1987); and Gribskov, M. & Devereux, J. (Ed.), Sequence Analysis Primer, M-Stockton Press, New York, (1991)). General methods used in determining the homology between two sequences include, but are not limited to, those disclosed in Martin, J. Bishop (Ed.), Guide to Huge Computers, Academic Press, San Diego, (1994); Carillo, H. & Lipman, D., SIAM J. Applied Math., 48: 1073 (1988), and the like.

Meanwhile, the activin used in the present invention may be any of an activin derived from the same biological species and an activin derived from a different biological species with respect to the pluripotent stem cells that will be cultured later. However, it is preferable that the activin and the pluripotent stem cells belong to species that are related to each other as closely as possible, and it is particularly preferable that the activin and the pluripotent stem cells be derived from the same biological species.

There are no particular limitations on the content of activin in the composition for pluripotent stem cell culture according to an aspect of the present invention, as long as the effects of the present invention are exerted, and the content can be appropriately determined by a person having ordinary skill in the art. When the content of activin in the composition is determined, reference may be made to the preferred activin concentration in the medium in the case where the relevant composition is used as the medium, as will be described below.

The composition for pluripotent stem cell culture may contain only one kind of activin, or may contain any combination of two or more kinds of activins. However, preferably, the composition for pluripotent stem cell culture contains at least activin A.

According to another aspect of the present invention, a medium for pluripotent stem cell culture which contains the composition for pluripotent stem cell culture described above is provided. This medium is such that even if supporting cells or serum is present in the medium, these components do not cause any hindrance to the culture of pluripotent stem cells. However, preferably, this medium is free of supporting cells and/or serum, and more preferably, the medium is free of supporting cells and serum.

That is, the medium for pluripotent stem cell culture according to an aspect of the present invention is preferably a medium which uses a cell culture minimum medium (CCMM) as a basal medium, to which differentiation inhibitory factors, serum replacement additives, antioxidants (for example, 2-mercaptoethanol (2-ME), dithiothreitol, and ascorbic acid), and the composition for pluripotent stem cell culture described above (that is, a composition containing activin) have been incorporated, and which is free of supporting cells and serum. The CCMM, differentiation inhibitory factors, serum replacement additives, antioxidants, and the composition for pluripotent stem cell culture related to the present invention described above are all known substances that can be artificially prepared, as will be described below. Therefore, the above-described medium constituted of these components is also preferable from the viewpoint that the contamination by unknown pathogens attributable to the use of biological components can be avoided.

In regard to the “cell culture minimum medium (CCMM)” used as the basal medium, there are no limitations on the specific formulation, as long as the medium enables undifferentiated proliferation of pluripotent stem cells when differentiation inhibitory factors, a serum replacement additives, antioxidants, and the composition for pluripotent stem cell culture according to the present invention are incorporated into the medium.

Conventionally, standard inorganic salts (zinc, iron, magnesium, calcium, potassium, and the like), vitamins, glucose, a buffer system, essential amino acids, and the like are added to the CCMM. Specific examples thereof include Dulbecco's Modified Eagle's Medium (DMEM), Minimal Essential Medium (MEM), Basal Medium eagle (BME), RPMI1640, F-10, F-12, α-Minimal Essential Medium (αMEM), Glasgow's Minimal Essential Medium (GMEM), and Iscove's Modified Dulbecco's Medium (IMDM), and commercially available products may also be used.

The CCMM may also contain 0.1 mM non-essential amino acids and 1 mM sodium pyruvate. The non-essential amino acids are a mixture of L-alanine, L-asparagine, L-aspartic acid, L-glutamic acid, glycine, L-proline, and L-serine, and for example, a product commercially available as a 10 mM liquid of MEM non-essential amino acids solution (manufactured by Life Technologies Japan, Ltd.) can be used. As the sodium pyruvate, for example, a product commercially available as a 100 mM liquid of MEM sodium pyruvate solution (manufactured by Life Technologies Japan, Ltd.) can be used.

The differentiation inhibitory factors are humoral factors released by supporting cells and pluripotent stem cells themselves, and inhibit the differentiation of undifferentiated cells. A representative differentiation inhibitory factor may be a leukemia inhibitory factor (LIF). Since the differentiation inhibitory factors are substances essentially present in living organisms, such substances can be collected from living organisms. However, in view of avoiding the contamination by pathogens, or from an economic viewpoint, it is preferable to use artificially synthesized factors. For example, in the case of a proteinous differentiation inhibitory factor such as LIF, it is preferable to use a recombinant differentiation inhibitory factor protein produced by genetic manipulation. Furthermore, another example of the differentiation factor maybe CHIR99021, which is a selective GSK-3β inhibitor. As will be shown in the Examples described below, according to a preferred embodiment of the composition for pluripotent stem cell culture according to the present invention, activin (for example, activin A) is used in combination with CHIR99021. Furthermore, according to another preferred embodiment, activin (for example, activin A) may also be used in combination with CHIR99021 and LIF.

Examples of the antioxidants that may be used include 2-mercaptoethanol, dithiothreitol, and ascorbic acid. Usually, 2-mercaptoethanol is used. These substances are commercially available and can be easily purchased.

A serum replacement additive means a substance which can support the proliferation of pluripotent stem cells when added to a serum-free medium. The serum replacement additive maybe a single substance, or may be a mixture. Specifically, the serum replacement additive is a preparation containing one or more components selected from albumin (for example, bovine serum albumin) or albumin replacement additives (for example, bovine pituitary extract, rice hydrolysate, fetal bovine albumin, egg albumin, human serum albumin, bovine embryo extract, and AlbuMAX I (registered trademark)), amino acids (for example, glycine, L-alanine, L-asparagine, L-cysteine, L-aspartic acid, L-glutamic acid, L-phenylalanine, L-histidine, L-isoleucine, L-lysine, L-leucine, L-glutamine, L-arginine, L-methionine, L-proline, L-hydroxyproline, L-serine, L-threonine, L-tryptophan, L-tyrosine, and L-valine), vitamins, transferrin or a transferrin replacement additives (for example, iron chelate compounds such as iron-chelates such as ethylenediaminetetraacetic acid, ethylene glycol-bis(β-aminoethylether)-N,N,N′,N′-tetraacetic acid, deferoxamine mesylate, dimercaptopropanol, diethylenetriaminepentaacetic acid, and trans-1,2-diaminocyclohexane-N,N,N′,N′-tetraacetic acid; ferric citrate chelates, and ferrous sulfate chelates), antioxidants (for example, reducing glutathione and ascorbic acid-2-monophosphate), insulin or insulin replacement additives (for example, zinc-containing compounds such as zinc chloride, zinc nitrate, zinc bromide, and zinc sulfate), collagen precursors (for example, L-proline, L-hydroxyproline, and ascorbic acid), and trace elements (for example, Ag⁺, Al³⁺, Ba²⁺, Cd²⁺, Co²⁺, Cr³⁺, Ge⁴⁺, Se⁴⁺, Br⁻, I⁻, Mn²⁺, F⁻, Si⁴⁺, V⁵⁻, Mo⁶⁺, Ni²⁺, Rb⁻, Sn²⁺, and Zr⁴⁺).

Meanwhile, examples of the serum replacement additive are described in detail in Japanese Patent Application National Publication (Laid-Open) No. 2001-508302 as “serum-free eukaryotic cell culture medium supplements,” and thus, the composition of the serum replacement additives may be appropriately determined by referring to the descriptions of the gazette. Representative serum replacement additives are sold by Life Technologies Japan, Ltd. as knock-out serum replacement additives (KSR), and are therefore easily available.

The LIF, 2-ME, and KSR described above are used in the medium usually at the final concentrations of 1 to 10,000 units/mL, 1 to 1000 μM, and 0.5% to 90% (v/v), respectively, and preferably at the final concentrations of 100 to 1000 units/mL, 10 to 100 μM, and 5% to 20%, respectively. There is no limitation on the concentration of activin in the medium (if plural activins are used, the total concentration), and the concentration can be appropriately determined to an extent that the effects of the present invention can be manifested, but the concentration is preferably 3 to 30 ng/mL, and more preferably 10 to 30 ng/mL. If the concentration of activin in the medium is 3 ng/mL or higher, when pluripotent stem cells are cultured using the medium, the undifferentiation potency of the pluripotent stem cells can be sufficiently maintained. On the other hand, if the concentration of activin in the medium is 30 ng/mL or lower, when pluripotent stem cells are cultured using the medium, the adverse effect of the addition of activin on the proliferative capacity of the pluripotent stem cells can be suppressed to the minimum.

The composition for pluripotent stem cell culture according to the present invention and the respective additive components described above may be added to a medium in amounts that make up the target final concentrations from the beginning, or may be added in amounts that are divided into two or more portions and finally make up the target concentrations. The pH of the medium can be adjusted to 7.0 to 8.2, and preferably to 7.3 to 7.9, usually by means of a hydrogen carbonate salt.

The composition for pluripotent stem cell culture and the medium for pluripotent stem cell culture according to the present invention can be respectively prepared in a solution form or in a dried form. In the case of a solution form, the composition and the medium may be provided as concentrated compositions (for example, 1× to 1000×), or may be suitably diluted at the time of use. Examples of the liquid used to dilute or dissolve the composition or medium that is in a solution form or a dried form, are water, an aqueous buffer solution, and a physiological saline solution, so that the liquid can be easily selected as necessary.

Preferably, the composition for pluripotent stem cell culture or the medium for pluripotent stem cell culture according to the present invention is a product that has been sterilized and prevented from contamination. The methods for sterilization include ultraviolet irradiation, heating sterilization, radioactive irradiation, filtration, and the like.

When it is intended to culture pluripotent stem cells using the technology according to the present invention, and to perform undifferentiated proliferation culture while maintaining the differentiation capacity of the pluripotent stem cells, it is desirable to culture pluripotent stem cells using the medium according to the present invention as described above, and preferably a medium prepared by incorporating a leukemia inhibitory factor, an antioxidant, a serum replacement additive, and the composition for pluripotent stem cell culture according to the present invention into a cell culture minimum medium, under the conventional culture conditions employed in the pertinent art.

In regard to the pluripotent stem cells, those derived from various animals such as mammals including human being, monkey, mouse, rat, hamster, rabbit, guinea pig, cattle, pig, dog, horse, cat, goat, and sheep, birds, and reptiles, maybe used. However, usually, pluripotent stem cells derived from a mammal are used. Specific examples of pluripotent stem cells include iPS cells, ES cells, EG cells, EC cells, APS cells, and MAP cells. There is no limitation on the number of pluripotent stem cells to be cultured, but the culturing method of the present invention is advantageous even from the viewpoint that the method enables a single pluripotent stem cell to be cultured to proliferate, and to thereby form a clonal cell population.

It is still acceptable that the pluripotent stem cells to be cultured are supporting cell-dependent per se, but supporting cell-independent pluripotent stem cells are preferred. As a technique for converting supporting cell-dependent pluripotent stem cells into supporting cell-independent cells, for example, the following treatment may be used. That is, subculture is repeated several times under the culturing conditions that do not use supporting cells, and cells suitable for such conditions may be selected.

The specific procedure of the method for culturing pluripotent stem cells according to the present invention can be carried out following the procedure and conditions, including culturing conditions, that are conventional in the pertinent art. For example, specific conditions can be appropriately determined by referring to the descriptions of Nakatsuji, Norio, ed.: Zikken Igaku (Experimental Medicine), Suppl. Vol. Experimental Course 4 in the Post-Genome Era, “Stem Cell and Clone Research Protocols,” Yodosha Co., Ltd. (2001); Hogan, G. et al., ed. Mouse Embryo Manipulations: A Laboratory Manual, Cold Spring Harbor Laboratory Press, Plainview, N.Y. (1994); Robertson, E. J., ed., Teratocarcinoma and Embryonic Stem Cells, A Practical Approach, IRL Press Oxford, UK (1987); and the like.

A representative subculture procedure and representative culture conditions are as follows. That is, in order to subculture iPS cells, first, full grown colonies of iPS cells are rinsed once or twice with a phosphate buffered saline (PBS), subsequently a sufficient amount of a trypsin-EDTA solution (0.25% trypsin-1 mM EDTA, in PBS) is added to cover the cell layer, which is left to stand for 5 minutes. Thereafter, PBS containing a trypsin inhibitor, or a basal culture fluid for iPS cell culture containing serum (CCMM+LIF+2-ME) is added, and cell clusters are separated by pipetting. From this cell suspension, cells are precipitated, usually by centrifugation. The supernatant is removed, and then precipitated cells are resuspended in a basal culture fluid for iPS cell culture containing serum or a serum replacement additive. A portion of this suspension is seeded into a supporting cell layer or on a gelatinated plastic plate, and the cells are cultured under the conditions of 37° C. and 5% CO₂.

According to an embodiment of the culturing method according to the present invention, the medium according to the present invention that has been warmed to 37° C., is placed on a plastic plate that has been gelatinated by treating with a 0.1% (w/v) gelatin solution, and pluripotent stem cells are seeded in the medium at a concentration of 10 to 1000 cells/cm₂ of the plate area. The plate is placed in a CO₂ incubator, and the cells are cultured under the conditions of 37° C. and 5% CO₂. When colonies grow fully, the cells are subcultured by seeding them in a fresh medium. During the subculture process, it is preferable to use PBS containing a trypsin inhibitor.

In regard to the lower density seeding conditions than those inducing undifferentiated proliferation of the pluripotent stem cells by the interaction between neighboring pluripotent stem cells, specifically, a seeding condition at a density of one cell/mm² or less may be a suitable example. A process of obtaining a clonal cell population from a single pluripotent stem cell on the occasion of the establishment of a uniform pluripotent stem cell lineage or the proliferation of pluripotent stem cells that have been subjected to genetic manipulation, of course, applies to this condition.

According to a preferred embodiment of the present invention, since neither supporting cells nor serum is used, screening of serum batches, and selection and culture of supporting cells, which are carried out in conventional culturing methods, can be eliminated. Furthermore, when the medium according to the present invention is used, a single supporting cell-independent pluripotent stem cells can be proliferated in an undifferentiated condition on a gelatin-coated plate under low density seeding conditions.

According to the culturing method according to the present invention, a clonal cell population can be formed by culturing and proliferating one pluripotent stem cell. This is advantageous in the case where a population of pluripotent stem cells having a modified genome is required, for example, in the case of producing a transgenic animal.

According to another aspect of the present invention, a composition for pluripotent stem cell culture containing activin is provided as a medium supplement. Furthermore, according to another aspect of the present invention, a medium for pluripotent stem cell culture containing the medium supplement is provided. Preferably, this medium is free of supporting cells and/or serum, and more preferably, the medium is free of supporting cells and serum. The medium can use a cell culture minimum medium as a basal medium. Also, this medium may further contain a differentiation inhibitory factor, a serum replacement additive, and an antioxidant.

According to still another aspect of the present invention, there is provided a method for culturing pluripotent stem cells, characterized by performing the culture of pluripotent stem cells in the presence of activin on the occasion of culturing pluripotent stem cells and proliferating undifferentiated pluripotent stem cells. In this culturing method, the culture can be carried out in the medium described above. Furthermore, in the culturing method, a single pluripotent stem cell is cultured, and thereby a clonal cell population thereof can be obtained. Also, pluripotent stem cells which do not undergo undifferentiated proliferation under the conditions free of supporting cells and/or serum and also free of the medium supplement described above, can be cultured in the medium described above, and thereby a clonal population of the pluripotent stem cells can be obtained. Preferably, in the culturing method, a single pluripotent stem cell is cultured in the medium described above, and thereby a clonal population thereof is obtained. Furthermore, in this culturing method, the pluripotent stem cells are preferably iPS cells, and the pluripotent stem cells are preferably cells derived from a mammal. It is more preferable that the pluripotent stem cells be human-derived cells. Meanwhile, according to still another aspect of the present invention, there are provided undifferentiated pluripotent cells retaining pluripotency, which have been proliferated by the culturing method described above.

According to still another aspect of the present invention, there is provided a method for culturing pluripotent stem cells, characterized by performing the culture of pluripotent stem cells in the presence of activin on the occasion of culturing pluripotent stem cells and establishing undifferentiated pluripotent stem cells. In the culturing method, the culture can be carried out in the medium described above. Furthermore, in the culturing method, the pluripotent stem cells are preferably iPS cells, and the pluripotent stem cells are preferably cells derived from a mammal. It is more preferable that the pluripotent stem cells be human-derived cells. According to still another aspect of the present invention, there are provided undifferentiated pluripotent stem cells retaining pluripotency, which have been established by the culturing method described above.

### EXAMPLES

Hereinafter, the present invention will be described in detail by way of Examples, but the present invention is not intended to be limited to the following Examples.

<<Cell culture>>

Cell culture was carried out using cells of strain HPS0001 201B7, which are human induced pluripotent stem cells (iPS cells), obtained from RIKEN BioResource Center, Cell Bank. The cell culture was carried out in a humidified chamber at 37° C. under the conditions of 5% CO₂ using, as a medium, Dulbecco minimum essential medium (DMEM)-F12 medium (manufactured by Sigma-Aldrich Japan K.K.) supplemented with a knockout serum replacement additive (KSR; manufactured by Life Technologies Japan, Ltd. (final concentration 20%), a 1/100 dilution of 100-times concentrated minimum essential amino acids (manufactured by Life Technologies Japan, Ltd.), 2 mM L-glutamine (manufactured by Life Technologies Japan, Ltd.), 0.1 mM 2-mercaptoethanol (manufactured by Sigma-Aldrich Japan K.K.), and 5 ng/mL of basic fibroblast growth factor (bFGF; manufactured by Wako Pure Chemical industries, Ltd.). At this time, 201B7 cell line was proliferated by co-culturing 201B7 cell line on cells of strain SNL76/7, which are mouse-derived fetal fibroblasts proliferated on a culture plate (manufactured by AGC Techno Glass Corp.) treated with mitomycin C (manufactured by Sigma-Aldrich Japan K.K.) and coated with gelatin (manufactured by Sigma-Aldrich Japan K.K.), and the 201B7 cell line was maintained. Also, at the time of subculturing, cells were collected using a CTK solution.

For the use in the experiment that will be described below, 201B7 cells were cultured using a feeder-free medium for primate ES cells (trade name: ReproFF, manufactured by ReproCELL, Inc.) as a medium, on a culture plate coated with Matrigel (manufactured by Becton, Dickinson and Company). Then, the cells were collected immediately before the experiment described below, using Accutase (manufactured by Innovative Cell Technologies, Inc.).

<<Formation of Embryoid Bodies Following Hanging Drop Culture Method>>

Dissociated 201B7 cells were cultured by a hanging drop culture method, at a density of 1000 cells per 30 μL of medium, and subculture and maintenance of 201B7 cell line was carried out (see Tomozawa M, Toyama Y, Ito C, et al., Hepatoblast-like cells enriched from mouse embryonic stem cells in medium without glucose, pyruvate, arginine, and tyrosine. Cell Tissue Res. 333: 17-27, 2008). At this time, culture was carried out using the same DMEM-F12 medium (containing bFGF) as that described above as a control, while in the Example, culture was carried out by adding activin A to a concentration of 100 ng/mL instead of bFGF.

<<Effect of Addition of Activin A on Differentiation of iPS Cells>>

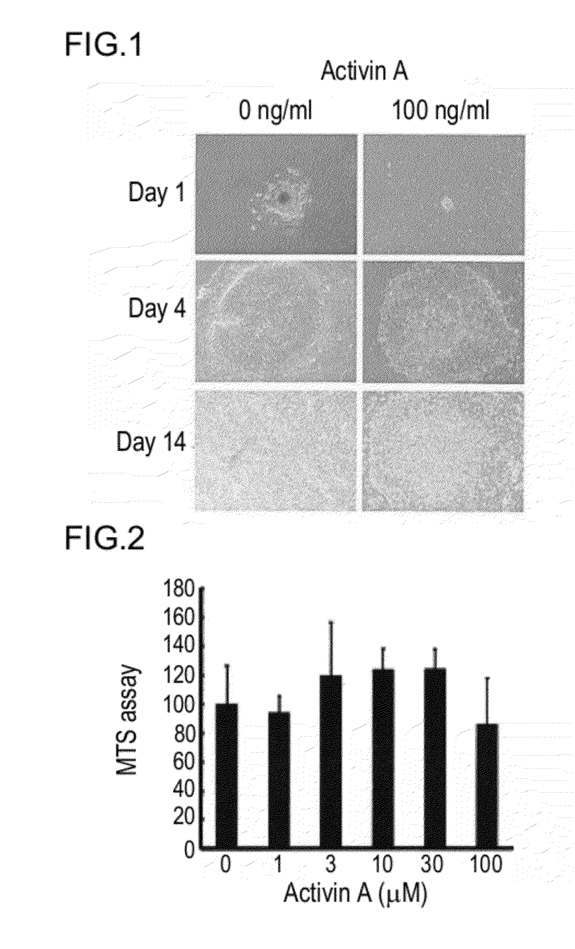

The hanging drop culture described above was carried out for 4 days, and then embryoid bodies thus obtained were transferred onto a Matrigel-coated plastic culture plate. Thereafter, the cells were respectively observed using an inverted tissue culture microscope (IMT-2) 1 day, 4 days, and 14 days after the initiation of culture. The results are shown in FIG. 1.

As shown in FIG. 1, in a culture system in which activin A was not added (0 ng/mL), differentiated cells appeared. On the contrary, in a culture system in which activin A was added to the medium (100 ng/mL), the embryoid bodies maintained a morphologically undifferentiated condition even for at least 14 days after the initiation of culture.

<<Evaluation of Cell Proliferative Capacity by MTS Assay>>

The proliferative capacity of 201B7 cells that had been cultured in the presence of activin A was evaluated by an MTS assay. The results are shown in FIG. 2.

As shown in FIG. 2, it could be seen that 201B7 cells had proliferative capacity at a concentration of added activin of 3 to 30 ng/mL.

<<Confirmation of Maintenance of Undifferentiation Potency of 201B7 Cells>>

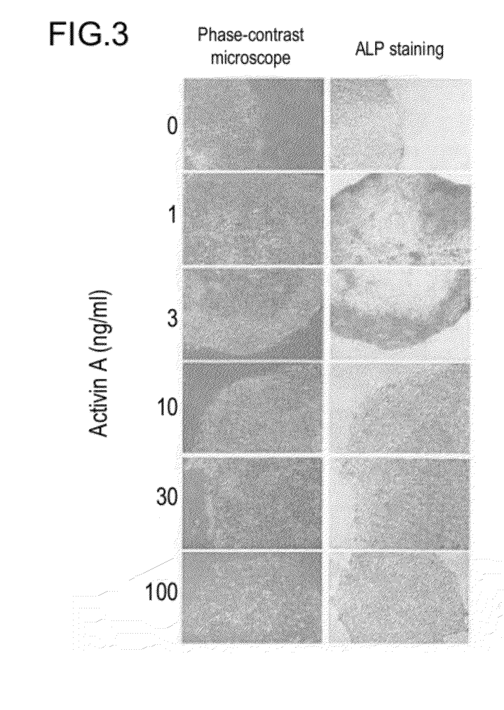

It was confirmed by alkaline phosphatase (ALP) staining that the 201B7 cells cultured in the presence of activin A maintained undifferentiation potency. The results are shown in FIG. 3. Meanwhile, the photographs on the left side of FIG. 3 are images observed with a phase contrast microscope, and the photographs on the right side are images observed after ALP staining.

First, in a culture system in which activin A was not added to the medium (0 ng/mL), differentiated cells appeared, and therefore, the cells were negative for ALP staining. In other words, it was verified that the cells had already lost undifferentiation potency. On the other hand, in a culture system in which activin A was added at a concentration of 1 ng/mL, the morphology of the 201B7 cells was slightly similar to fibroblasts, and the cells were positive for ALP staining. However, a possibility was suggested that the cells might not perfectly maintain undifferentiation potency. Furthermore, in systems in which activin A was added to concentrations of 10 ng/mL, 30 ng/mL, and 100 ng/mL, the morphology of the cells maintained an undifferentiated condition, and the cells were also positive for ALP staining. Therefore, we, inventors, thought that iPS cells can maintain undifferentiation potency at a concentration of added activin A of 10 to 100 ng/mL.

From the results described above, a conclusion can be drawn that as a technique of culturing pluripotent stem cells such as iPS cells while maintaining their undifferentiation potency, it is more preferable to add activin A at a concentration of 3 to 30 ng/mL, even from the viewpoint of maintaining proliferative capacity.

<<Subculture>>

Subculture was carried out by adding the following various agents to iPSm(−).

SU5402 (FGFR1 inhibitor; manufactured by Wako Pure Chemical Industries, Ltd.): 2 μM

SC-1 (RasGAP, ERK1 inhibitor; manufactured by Wako Pure Chemical Industries, Ltd.): 1 μM

Activin A (manufactured by R & D Systems, Inc.): 10 ng/mL

CHIR99021 (GSK-3β inhibitor; manufactured by Wako Pure Chemical Industries, Ltd.): 2 μM

Human leukemia inhibitory factor (LIF; manufactured by Sigma-Aldrich Japan K.K.): 1000 U/mL

Basic fibroblast growth factor (bFGF; manufactured by Wako Pure Chemical Industries, Ltd.): 5 ng/mL.

201B7 cells were seeded on a Matrigel-coated 6-well plate, and the above-described agents were added alone or simultaneously to iPSm(−). At the time point when the cells reached 70% confluency, the cells were collected with Accutase and were seeded on a new Matrigel-coated 6-well plate. The medium was exchanged in every 48 hours. The cell morphology was observed with a phase contrast microscope, and at the time point when differentiated cells were recognized or the cells no longer proliferated, the subculture process was terminated. Also, the culturing process was terminated after 12 subcultures.

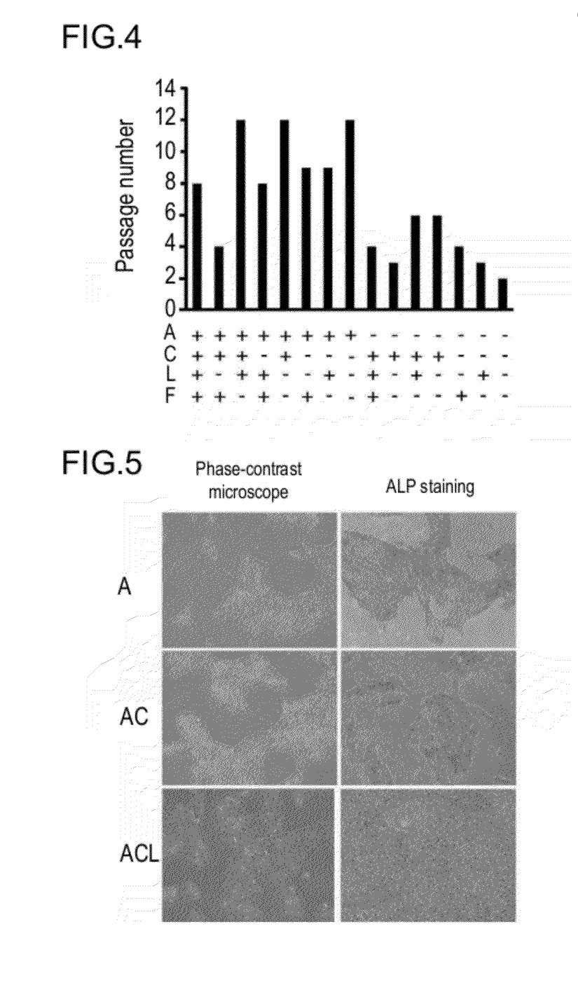

In regard to the changes in the number of subcultures due to the form of addition of the agents described above, the results are shown in FIG. 4. In FIG. 4, reference symbol “A” represents activin A (10 ng/mL), “C” represents CHIR99021 (2 μM), “L” represents LIF (1000 U/mL), and “F” represents basic fibroblast growth factor (5 ng/mL). Furthermore, images observed with a phase contrast microscope (at the 12th passage) are shown on the left side of FIG. 5. In FIG. 5, reference symbol “A” represents activin A alone, “AC” represents a combination of activin A+CHIR99021, and “ACL” represents a combination of activin A+CHIR99021+LIF.

As shown in FIG. 4, in a culture system in which activin A was added, the number of subcultures was larger than that in a culture system which was not added with activin A. Particularly, in the culture systems with activin A alone, a combination of activin A+CHIR99021, and a combination of activin A+CHIR99021+LIF, the passage number exceeded 12.

From the results illustrated above, it can be seen that when culture is carried out using a medium containing the composition for pluripotent stem cell culture according to the present invention, subculture can be repeated.

<<Alkaline Phosphatase Staining>>

Among the cells that had been subcultured as described above, those subcultured in the presence of activin A alone (A), a combination of activin A+CHIR99021 (AC), and a combination of activin A+CHIR99021+LIF (ACL) were subjected to alkaline phosphatase staining as described above, at the 12th passage. The results are shown in the photographs on the left side of FIG. 5.

As shown in the photographs, the cells subcultured in the presence of activin A alone (A), a combination of activin A+CHIR99021 (AC), and a combination of activin A+CHIR99021+LIF (ACL) were all positive for ALP staining. However, in regard to the combination of ACL, the staining was slightly weaker in intensity. It is known that cells having pluripotency are positive for ALP staining (Goldstein D J et al. Expression of alkaline phosphatase loci in mammalian tissues. Proc. Natl. Acad. Sci. USA, 77(5): 2857-2860, 1980), and therefore, it can be considered that those cells that are positive for ALP staining as described above could be repeatedly subcultured while maintaining pluripotency.

<<Immunostaining>>

Each of the cells at the 11th passage was seeded on a Matrigel-coated 4-well slide glass, and subculture was continued. When the cells reached 70% confluency, the medium was removed, and the cells were washed once with PBS. The cells were immobilized with 4% para-formaldehyde, and then were washed three times with PBS. Meanwhile, in the case of staining for Oct3/4 and Nanog, a permeabilization solution (0.2% Triton X-100 (PBS solution)) was added to the cells, and the cells were left to stand for 15 minutes at room temperature. Subsequently, the cells were washed three times for 5 minutes with a washing buffer (2% FBS (PBS solution)).

Thereafter, primary antibodies diluted with the washing buffer were added to the cells, and the cells were left to stand overnight at 4° C. Meanwhile, the types of the various primary antibodies and the dilution ratios are as follows.

Mouse anti-Oct3/4 antibody (BD Transduction Laboratories): 200 times

(Primary Antibody)

Rabbit anti-Nanog antibody (manufactured by ReproCELL Inc.): 1000 times

Mouse anti-SSEA4 antibody (manufactured by Chemicon Corporation): 200 times

Mouse anti-TRA-1-60 antibody (manufactured by Chemicon Corporation): 200 times

Subsequently, the cells were diluted three times for 5 minutes with the washing buffer, and then secondary antibodies diluted with the washing buffer were added. The cells were left to stand for one hour at 4° C. The types of the various secondary antibodies and the dilution ratios are as follows.

(Secondary Antibody)

HRP anti-mouse (manufactured by GE Healthcare): 500 times

HRP anti-rabbit (manufactured by GE Healthcare): 500 times

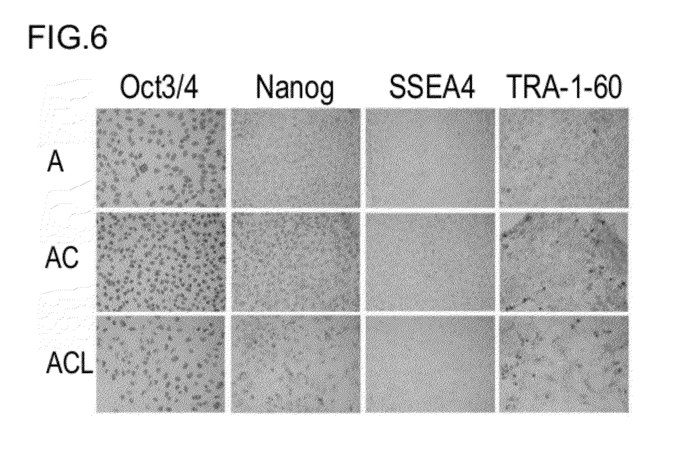

Thereafter, the cells were washed three times for 5 minutes with the washing buffer, and the cells were made to develop color using Liquid DAB+Substrate Chromogen System (DAKO). Subsequently, the cells were stained with Mayer's Hematoxylin (manufactured by Muto Pure Chemicals Co., Ltd.) for 10 seconds, washed with water for 30 minutes, and were encapsulated. The results are shown in FIG. 6.

As shown in FIG. 6, the nuclei of the cells subcultured in the presence of A, AC, and ACL were all positively stained by the anti-Oct3/4 antibody. Furthermore, in the case of the anti-Nanog antibody, the nuclei of the cells subcultured in the presence of AC were most strongly positively stained. Furthermore, when the cells were stained by the anti-SSEA4 antibody or the anti-TRA-1-60 antibody, the cytoplasm of the cells subcultured in the presence of AC was most strongly positively stained in both cases. It is known that these antibodies are generally used as indicators of pluripotency (Takahashi et al. Induction of pluripotent stem cells from adult human fibroblasts by defined factors. Cell, (2007) 131, 861-872). Therefore, we, inventors, thought that those cells that are positively stained in immunostaining by the various antibodies described above, are able to be repeatedly subcultured while maintaining pluripotency.

1. A composition for pluripotent stem cell culture, comprising activin.

2. The composition according to claim 1, wherein the composition is a medium supplement.

3. The composition according to claim 1, wherein the composition is intended for proliferating pluripotent stem cells while maintaining an undifferentiated condition of the pluripotent stem cells.

4. The composition according to claim 1, wherein the activin is activin A.

5. A medium for pluripotent stem cell culture, comprising the composition according to claim 1.

6. The medium according to claim 5, comprising activin at a concentration of 3 to 30 ng/mL.

7. The medium according to claim 5, wherein the medium is free of supporting cells and/or serum.

8. The medium according to claim 7, wherein the medium is free of supporting cells and serum.

9. The medium according to claim 5, wherein the medium is a cell culture minimum medium.

10. A method for culturing pluripotent stem cells for proliferating or establishing pluripotent stem cells while maintaining an undifferentiated condition of the pluripotent stem cells, the method comprising performing the culture in the presence of activin.

11. The method according to claim 10, wherein the culture is carried out in a medium containing activin.

12. The method according to claim 10, wherein the pluripotent stem cells are induced pluripotent stem cells.

13. The method according to claim 10, wherein the pluripotent stem cells are cells derived from a mammal.

14. The method according to claim 13, wherein the pluripotent stem cells are human-derived cells.

15. A method for preparing a pluripotent stem cell clonal population in an undifferentiated condition, the method comprising culturing undifferentiated pluripotent stem cells in the presence of activin.

16. A method for preparing a pluripotent stem cell clonal population in an undifferentiated condition, the method comprising isolating pluripotent stem cells in an undifferentiated condition from a living organism, and culturing undifferentiated pluripotent stem cells in the presence of activin.

17. The method according to claim 15, wherein the culture is carried out in a medium containing activin.

18. The method according to claim 15, wherein a single pluripotent stem cell is cultured, and thereby a clonal population of the cells is obtained.

19. The method according to claim 15, wherein pluripotent stem cells that are under lower density seeding conditions than those inducing undifferentiated proliferation of the pluripotent stem cells by the interaction between neighboring pluripotent stem cells, are cultured in a medium containing activin and free of supporting cells and/or serums, and thereby a clonal population of the cells is obtained.

20. The method according to claim 15, wherein a single pluripotent stem cell is cultured in a medium containing activin and free of supporting cells and/or serums, and a clonal population of the cell is obtained.

21. The method according to claim 15, wherein the pluripotent stem cells are induced pluripotent stem cells.

22. The method according to claim 15, wherein the pluripotent stem cells are cells derived from a mammal.

23. The method according to claim 22, wherein the pluripotent stem cells are human-derived cells.

24-26. (canceled)
